(19) United States
(12) Patent Application Publication
(10) Pub. No.: US 2024/0043434 A1
(43) Pub. Date: Feb. 8, 2024

(54) COCRYSTALS OF UPADACITINIB

(71) Applicant: AbbVie Inc., North Chicago, IL (US)

(72) Inventor: Alessandra Mattei, Chicago, IL (US)

(21) Appl. No.: 18/482,794

(22) Filed: Oct. 6, 2023

Related U.S. Application Data
(63) Continuation of application No. PCT/US22/74596, filed on Aug. 5, 2022.
(60) Provisional application No. 63/171,855, filed on Apr. 7, 2021.

(51) Int. Cl. C07D 487/14

(52) U.S. Cl. CPC C07D 487/14; C07B 2200/13

(57) ABSTRACT

The disclosure relates to cocrystals of solid state forms of (3S,4R)-3-ethyl-4-(3H-imidazo[1,2-a]pyrrolo[2,3-e]-pyrazin-8-yl)-N-(2,2,2-trifluoroethyl)pyrrolidine-1-carboxamide (Compound 1). Specifically, the present disclosure relates to cocrystals of Compound 1 and a suitable coformer, such as a substituted benzoic acid.

### CROSS-REFERENCE TO RELATED APPLICATIONS

This application is a continuation of International Application No. PCT/US2022/071596, filed Apr. 7, 2022, and claims priority to U.S. Provisional Application No. 63/171,855, filed Apr. 7, 2021, the disclosure of each of which are hereby incorporated by reference in their entireties.

### TECHNICAL FIELD

The present disclosure relates to solid state forms of (3S,4R)-3-ethyl-4-(3H-imidazo[1,2-a]pyrrolo[2,3-e]-pyrazin-8-yl)-N-(2,2,2-trifluoroethyl)pyrrolidine-1-carboxamide (upadacitinib; “Compound 1”). Specifically, the present disclosure relates to cocrystals comprising Compound 1 and one or more suitable coformers.

### BACKGROUND

Upadacitinib, marketed under the brand name Rinvoq™, is a selective Janus kinase 1 (“JAK-1”) inhibitor approved by the FDA for the treatment of moderately to severely active rheumatoid arthritis in adults where methotrexate did not work or could not be tolerated. The chemical name of upadacitinib is (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide, referred to herein as “Compound 1”, which was first disclosed in International Application WO2011/068881A1, which application is herein incorporated by reference in its entirety. Compound 1 has the structure:

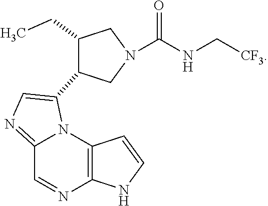

The variety of possible solid state forms of any particular active pharmaceutical ingredient (API; e.g., Compound 1) creates the potential for diversity in physical and chemical properties for the API. The discovery and selection of solid state forms are of great importance in the development of an effective, stable, and marketable pharmaceutical product. These physical and chemical properties include, but are not limited to: (1) packing properties such as molar volume, bulk density and hygroscopicity, (2) thermodynamic properties such as melting temperature, vapor pressure and solubility, (3) kinetic properties such as dissolution rate and stability (including stability at ambient conditions, especially to moisture and under storage conditions), (4) surface properties such as surface area, wettability, interfacial tension and shape, (5) mechanical properties such as hardness, tensile strength, compactibility, handling, flow and blend; and (6) filtration properties. These properties can affect, for example, the processing and storage of the compound and pharmaceutical compositions comprising the compound.

Solid state forms of Compound 1 that improve upon one or more properties relative to other solid state forms of the compound are desirable. Accordingly, there remains a need for additional solid state forms of Compound 1 that have an acceptable balance of properties including chemical stability, thermal stability, solubility, hygroscopicity, and/or particle size, milling properties, and formulation feasibility (including stability with respect to pressure or compression forces during tableting) and that can be used in the preparation of pharmaceutically acceptable solid dosage forms of Compound 1.

### SUMMARY OF THE DISCLOSURE

The present disclosure generally provides solid state forms of (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (“Compound 1”). Different solid state forms of the same compound (e.g., Compound 1) may have different crystal packing, thermodynamic, spectroscopic, kinetic, surface, and mechanical properties. For example, different solid state forms may exhibit greater compressibility and/or density properties which provide more desirable characteristics for formulation and/or product manufacturing. Particular solid state forms may also have different dissolution rates, thereby providing different pharmacokinetic parameters, allowing for specific solid state forms to be selected to achieve specific pharmacokinetic parameters. Such parameters may include, but are not limited to, solubility, dissolution, bioavailability, stability, Cₘₐₓ, Tₘₐₓ, and exposure (i.e., area under the curve; AUC). Pharmaceutical cocrystals are attractive because they offer multiple opportunities to modify the chemical and/or physical properties of an API without making or breaking covalent bonds.

Accordingly, in a first aspect is provided a cocrystal comprising (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (Upadacitinib; “Compound 1”) and a coformer, wherein the coformer is an aryl carboxylic acid.

In some embodiments, the aryl carboxylic acid is a substituted benzoic acid.

In some embodiments, the substituted benzoic acid has a structure of Formula (I)

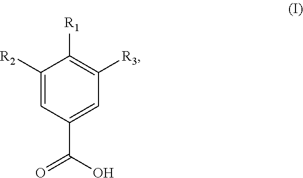

wherein:
   R₁ is —NHC(O)CH₃ or —OH;
   R₂ is —H, —OH, or NO₂; and
   R₃ is —H, —OH, or NO₂.

In some embodiments, R₁ is —NHC(O)CH₃, and R₂ and R₃ are each H. In some embodiments, R₁ is OH, and R₂ and R₃ are each H. In some embodiments, R₁ is OH, R₂ is —OH, and R₃ is H. In some embodiments, R₁, R₂ and R₃ are —OH. In some embodiments, R₁ is OH, R₂ is —NO₂, and R₃ is H.

In some embodiments, a molar ratio of Compound 1 to coformer is from about 5:1 to about 1:5. In some embodiments, the molar ratio is from about 2:1 to about 1:2, or from about 1:1.5 to about 1.5:1. In some embodiments, the molar ratio is about 1:1.

In some embodiments, the cocrystal is a solvate. In some embodiments, the cocrystal solvate comprises acetonitrile. In some embodiments, the cocrystal solvate further comprises water.

In some embodiments, the cocrystal has one or more of reduced aqueous solubility, reduced dissolution, enhanced bioavailability, enhanced stability, increased Cₘₐₓ, increased or decreased Tₘₐₓ, increased half-life, increased AUC, enhanced processability, and reduced hygroscopicity, relative to Compound 1 as a free base or a salt, including solvates, hydrates, and polymorphs of any thereof.

In another aspect is provided a cocrystal comprising (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (Compound 1) and 4-acetamidobenzoic acid in a molar ratio of approximately 1:1.

In some embodiments, the cocrystal is an acetonitrile solvate. In some embodiments, the acetonitrile solvate cocrystal is a hydrate.

In some embodiments, the acetonitrile solvate cocrystal hydrate has a powder x-ray diffraction pattern characterized by peaks at 5.1±0.2, 10.2±0.2, and 12.5±0.2 degrees two theta when measured at about 25° C. with monochromatic Kα1 radiation λ=1.540562 Å.

### BRIEF DESCRIPTION OF THE DRAWINGS

FIG. 1 is representative powder X-ray diffraction pattern corresponding to a non-limiting embodiment of a cocrystal according to the present disclosure comprising Compound 1 ((3S,4R)-3-ethyl-4-(3H-imidazo[1,2-a]pyrrolo[2,3-e]pyrazin-8-yl)-N-(2,2,2-trifluoroethyl)pyrrolidine-1-carboxamide; upadacitinib) and 4-acetamidobenzoic acid.

FIG. 2 is representative powder X-ray diffraction pattern corresponding to a non-limiting embodiment of a cocrystal according to the present disclosure comprising Compound 1 and 4-acetamidobenzoic acid.

FIG. 3 is representative powder X-ray diffraction pattern corresponding to a non-limiting embodiment of a cocrystal according to the present disclosure comprising Compound 1 and 4-acetamidobenzoic acid.

FIG. 4 is representative powder X-ray diffraction pattern corresponding to a non-limiting embodiment of a cocrystal according to the present disclosure comprising Compound 1 and 4-hydroxybenzoic acid.

FIG. 5 is representative powder X-ray diffraction pattern corresponding to a non-limiting embodiment of a cocrystal according to the present disclosure comprising Compound 1 and 4-hydroxybenzoic acid.

FIG. 6 is representative powder X-ray diffraction pattern corresponding to a non-limiting embodiment of a cocrystal according to the present disclosure comprising Compound 1 and 4-hydroxy-3-nitrobenzoic acid.

FIG. 7 is representative powder X-ray diffraction pattern corresponding to a non-limiting embodiment of a cocrystal according to the present disclosure comprising Compound 1 and 4-hydroxy-3-nitrobenzoic acid.

FIG. 8 is representative powder X-ray diffraction pattern corresponding to a non-limiting embodiment of a cocrystal according to the present disclosure comprising Compound 1 and 3,4-dihydroxybenzoic acid.

FIG. 9 is representative powder X-ray diffraction pattern corresponding to a non-limiting embodiment of a cocrystal according to the present disclosure comprising Compound 1 and 3,4-dihydroxybenzoic acid.

### DETAILED DESCRIPTION

The present disclosure generally provides cocrystals comprising (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (“Compound 1”) and one or more coformers.

According to the present disclosure, it is believed, without wishing to be bound by theory, that when Compound 1 and a selected coformer are allowed to form cocrystals, the resulting cocrystals may give rise to improved properties as compared to other solid state forms of Compound 1 (including amorphous or crystalline forms, which may be a free base or salt, or hydrate or solvate of any thereof). Such improved properties may include one or more of: solubility, dissolution, bioavailability, stability, Cₘₐₓ, Tₘₐₓ, processability, longer lasting therapeutic plasma concentration, hygroscopicity, and crystalline form. Suitable coformers are described herein below, along with methods for preparation and characterization thereof, and select properties of such cocrystals.

### Definitions

With respect to the terms used in this disclosure, the following definitions are provided.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below. The term “alkyl” refers to straight chained or branched hydrocarbons which are completely saturated. For purposes of exemplification, which should not be construed as limiting the scope of this invention, examples of alkyls include methyl, ethyl, propyl, isopropyl, butyl, pentyl, hexyl, and isomers thereof. An alkyl group may be substituted or unsubstituted.

The term “cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a mono-, bi-, or tricyclic aromatic hydrocarbon radical. Examples of aryl groups include, but are not limited to, phenyl and naphthyl. An aryl group can be unsubstituted or substituted.

“Heteroaryl” as used herein refer to an aromatic ring system in which one or more ring atoms is a heteroatom, e.g. nitrogen, oxygen, and sulfur. The heteroaryl group comprises up to 20 carbon atoms and from 1 to 3 heteroatoms selected from N, O, and S. A heteroaryl may be a monocycle having 5 or 6 ring members (for example, 1 to 5 carbon atoms and 1 to 3 heteroatoms selected from N, O, and S), or a bicycle having 7 to 10 ring members (for example, 4 to 9 carbon atoms and 1 to 3 heteroatoms selected from N, O, and S). Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Heteroaryl groups can be unsubstituted or substituted.

The term “substituted” as used herein and as applied to any of the above alkyl, cycloalkyl, aryl, and, heteroaryl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion.

The term “solid state” when used herein refer to a physical form comprising Compound 1 which is not predominantly in a liquid or a gaseous state. As used herein, the term “solid state” encompasses semi-solids. Solid state forms may be crystalline, amorphous, partially crystalline, partially amorphous, or mixtures of any thereof.

The term “amorphous” as applied to a compound refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”).

The term “crystalline” as applied to a compound refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”). In some embodiments, a crystalline form of a substance (e.g., a cocrystal comprising Compound 1) may contain less than about 50%, 40%, 30%, 20%, 10%, 5%, or 1% of one or more amorphous form(s) on a weight basis. In some embodiments, the crystalline form may be substantially free of amorphous forms, such as less than about 1%, less than about 0.1%, less than about 0.01%, or even 0% of amorphous forms on a weight basis.

The term “crystalline purity” means the crystalline purity of a compound with regard to a particular crystalline form of the compound as determined by the powder X-ray diffraction analytical methods described in this application. In some embodiments, a crystalline form of a substance (e.g., a cocrystal comprising Compound 1) may be substantially free of other crystalline forms. In some embodiments, the crystalline form may contain at least about 50%, 60%, 70%, 80%, 90%, 95%, 99%, 99.5%, 99.9%, or even 100% of one specific crystalline form on a weight basis.

The term “crystallization” as used throughout this application can refer to crystallization and/or recrystallization depending upon the applicable circumstances relating to the preparation of the compound.

The term “pharmaceutically acceptable” (such as in the recitation of a “pharmaceutically acceptable salt” refers to a material that is compatible with administration to a human subject, e.g., the material does not cause an undesirable biological effect and/or is, within the scope of sound medical judgment, suitable for use in contact with the tissues of humans and other mammals without undue toxicity, irritation, allergic response and the like, and are commensurate with a reasonable benefit/risk ratio. Examples of pharmaceutically acceptable salts are described in “Handbook of Pharmaceutical Salts: Properties, Selection, and Use” by Stahl and Wermuth (Wiley-VCH, Weinheim, Germany, 2002). Examples of pharmaceutically acceptable excipients are described in the “Handbook of Pharmaceutical Excipients,” Rowe et al., Ed. (Pharmaceutical Press, 7th Ed., 2012).

As used herein, the term “cocrystal” refers to a crystalline solid made up of two or more unique chemical species in the same crystal lattice, in a defined stoichiometric ratio, and that possesses distinct physical, crystallographic and spectroscopic properties when compared to the chemical species individually. Present cocrystals comprise the API Compound 1 and one or more coformers as described herein below.

A cocrystal is distinct from a “salt,” which comprises charged-balanced charged species. The species making up a cocrystal typically are neutral, and are generally held together by weak, freely reversible, non-covalent interactions. The weak interaction is defined as neither ionic bond interaction nor covalent bond interaction, and include hydrogen bonding, van der Waals forces, π-π interactions and halogen bond interactions. Cocrystals can generally be distinguished from salts by the absence of a proton transfer between the chemical species.

### Coformers

A cocrystal of Compound 1 as described herein comprises Compound 1 and at least one additional chemical species, generally referred to as a “cocrystal former” or “coformer.” The coformer may be H-bonded directly to Compound 1 or may be H-bonded to an additional molecule (a second coformer) which is H-bonded to Compound 1. Other modes of molecular recognition may also be present, including π-π interactions, guest-host complexation, and van der Waals interactions. Of the interactions listed above, hydrogen bonding is generally the dominant interaction in the formation of the present cocrystals, whereby a non-covalent bond is formed between a hydrogen bond donor of one of the chemical species and a hydrogen bond acceptor of the other.

In certain embodiments, the non-covalent forces holding the coformer and Compound 1 together are selected form the group consisting of pi-stacking, guest-host complexation, van der Waals interactions, and combinations thereof. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads.

As used herein, reference to “a coformer” or “the coformer” includes the possibility of more than one, such as two, or even three, different coformers; however, for simplicity, such multiple coformers are referred to herein in the singular.

The coformer of the present cocrystal may be any pharmaceutically acceptable molecule(s) that forms a cocrystal with Compound 1. Advantageously (although not necessarily), coformers that are combined with Compound 1 to form cocrystals are selected from those “Generally Regarded As Safe” (“GRAS”) by the U.S. Food and Drug Administration. The GRAS list contains about 2500 relevant compounds certain of which may be suitable as coformers. It is noted that certain coformers as described herein may contain one or more chiral centers, which may be either of the (R)- or (S)-configuration, or which may comprise a mixture thereof. Certain coformers as described herein may be geometric isomers, including but not limited to cis and trans isomers across a double bond.

In some embodiments, the coformer is an organic acid. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). In some embodiments, the organic acid is a solid organic acid, meaning the organic acid is in a solid physical form at typical room temperature, for example, at about 15 to about 25° C. (i.e., having a melting point greater than about 15 or greater than about 25° C.).

In some embodiments, the organic acid is a carboxylic acid. The carboxylic acid functional group may be attached to any alkyl, cycloalkyl, aryl, or heteroaryl group having, for example, from one to twenty carbon atoms (C₁-C₂₀).

In some embodiments, the carboxylic acid is an alkyl or cycloalkyl carboxylic acid. Examples of suitable alkyl and cycloalkyl carboxylic acid include, but are not limited to, acetic acid, 2,2-dichloroacetic acid, butyric acid, propionic acid, pyruvic acid, isobutyric acid, 2-ethylbutyric acid, 3-methylbutanoic acid, tiglic acid, valeric acid, levulinic acid, valproic acid, hexanoic acid, pivalic acid, 3-cyclopentylpropionic acid, 1,2,2-trimethyl-1,3-cyclopentanedicarboxylic acid, cyclohexanecarboxylic acid, cyclohexylacetic acid, octanoic acid, decanoic acid, lauric acid, tetradecanoic acid, oleic acid, palmitic acid, sorbic acid, stearic acid, (+)-camphoric acid, 10-undecylenic acid, orotic acid, ethylenediaminetetraacetic acid, citric acid, alpha-hydroxypropionic acid, tartaric acid, glycolic acid, ascorbic acid, lactic acid, malic acid, galactaric acid, glucoheptonic acid, gluconic acid, glucuronic acid, and lactobionic acid.

In some embodiments, the alkyl carboxylic acid is a dicarboxylic acid. Examples of suitable alkyl dicarboxylic acids include, but are not limited to, oxalic acid, malonic acid, succinic acid, glutaric acid, adipic acid, ketoglutaric acid, fumaric acid, maleic acid, and sebacic acid.

In some embodiments, the carboxylic acid is an amino acid. Examples of suitable amino acids include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In some embodiments, the organic acid is an aryl carboxylic acid or heteroaryl carboxylic acid. Examples of suitable aryl or heteroaryl carboxylic acids include, but are not limited to, cinnamic acid, 3-phenylpropionic acid, diphenylacetic acid, mandelic acid, nicotinic acid, 2-furancarboxylic acid, phenylacetic acid, phenoxyacetic acid, and pamoic acid.

In some embodiments, the aryl carboxylic acid is a substituted or unsubstituted benzoic acid. Examples of suitable benzoic acids include, but are not limited to, benzoic acid, 2-hydroxybenzoic acid, 3-hydroxybenzoic acid, 4-hydroxybenzoic acid, 4-aminobenzoic acid, 4-aminosalicylic acid, 3-acetamidobenzoic acid, 4-acetamidobenzoic acid, benzene-1,3-dicarboxylic acid, benzene-1,3,5-tricarboxylic acid, o-toluic acid, m-toluic acid, p-toluic acid, 2,4-dihydroxybenzoic acid, 2,5-dihydroxybenzoic acid, m-methoxybenzoic acid, anisic acid, acetylsalicylic acid, 1-hydroxy-2-naphthoic acid, terephthalic acid, 2-mercaptobenzoic acid, sulfosalicylic acid, gallic acid, gentisic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluorobenzoic acid, 3-fluorobenzoic acid, 4-fluorobenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluorobenzoic acid, 2,5-difluorobenzoic acid, 2,6-difluorobenzoic acid, 3,4,5-trifluorobenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, 2,3,4,5-tetrafluoro-6-hydroxybenzoic acid, 2,3-dihydroxybenzoic acid (pyrocatechuic acid/hypogallic acid), 2,4-dihydroxybenzoic acid (β-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihdroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, 5-amino-3-chloro-2,4-dihydroxybenzoic acid, 2,3,4-trihydroxybenzoic acid, 2,4,5-trihydroxybenzoic acid, 2,4,6-trihydroxybenzoic acid (phloroglucinol carboxylic acid), 3,4,5-trihydroxybenzoic acid (gallic acid), 1,2,3-benzenetricarboxylic acid, 1,2,4-benzenetricarboxylic acid (trimellitic acid), 1,2-benzenedicarboxylic acid (pthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid, 1,2,3,4-benzenetetracarboxylic acid (mellophanic acid), and 1,2,4,5-benzenetetracarboxylic acid (pyromellitic acid).

In some embodiments, the coformer is a benzoic acid substituted at one or more positions, the substituents selected independently for each occasion from the group consisting of hydrogen bond donors, electron withdrawing groups, and electron donating groups. In some embodiments, a difference in a pKa (Δ pKa) value between the benzoic acid and Compound 1 is less than about 1.

In some embodiments, the coformer is a benzoic acid according to Formula I,

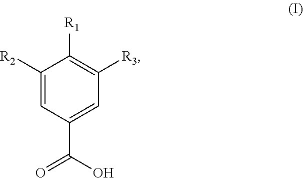

wherein:
   R₁ is —NHC(O)CH₃ or —OH;
   R₂ is —H, —OH, or NO₂; and
   R₃ is —H, —OH, or NO₂.

In some embodiments, R₁ is —NHC(O)CH₃, and R₂ and R₃ are each H.

In some embodiments, R₁ is OH, and R₂ and R₃ are each H.

In some embodiments, R₁ is OH, R₂ is —OH, and R₃ is H.

In some embodiments, R₁, R₂ and R₃ are —OH.

In some embodiments, R₁ is OH, R₂ is —NO₂, and R₃ is H.

In some embodiments, the conformer is selected from the group consisting of 4-acetamidobenzoic acid, 4-hydroxybenzoic acid, 4-hydroxy-3-nitrobenzoic acid, and gallic acid. In some embodiments, the conformer is 4-acetamidobenzoic acid.

### Ratios

The ratio of Compound 1 to coformer may be stoichiometric or non-stoichiometric. For example, various ratios of Compound 1 to coformer are possible, such as from about 5:1 to about 1:5, or from about 2:1 to about 1:2, or from about 1:1.5 to about 1.5:1. In some embodiments, the ratio is about 5:1, about 4:1, about 3:1, about 2:1, about 1.5:1, about 1:1, about 1:1.5, about 1:2, about 1:3, about 1:4, or about 1:5. In some embodiments, the ratio is stoichiometric, such as about 1:1. One of skill in the art will recognize that such a molar ratio of components provides information as to the general relative quantities of the components of the crystalline form. However, in many cases, the molar ratio may vary by ±20% from a stated range. For example, with respect to the present disclosure, a molar ratio of 1:1 should be understood to include the ratios 1:0.8 and 1:1.2, as well as all of the individual ratios in between.

### Solvates

In certain embodiments, the cocrystals may include one or more solvate molecules in the crystalline lattice, i.e., solvates of cocrystals, or a cocrystal further comprising a solvent or compound that is a liquid at room temperature. The one or more solvent molecules may in some embodiments include water, in which case the cocrystal is referred to as a “hydrate.” As used herein, the terms “hydrate” and “solvate” refers to inclusion in the crystal lattice of a stoichiometric or non-stoichiometric amount of water or solvent, respectively, bound by non-covalent intermolecular forces. In some embodiments, the solvent molecule is acetonitrile. In some embodiments, the solvent molecule is water. In some embodiments, the solvent molecule is both acetonitrile and water.

### Cocrystal Preparation

Cocrystals as disclosed herein comprising Compound 1 may be prepared according to a number of different methods. Suitable techniques for cocrystal formation are disclosed in, for example, Karimi-Jafari et al., Crystal Growth and Design 2018, 18, 6370-6387, which is incorporated by reference herein in its entirety. Generally, the methods comprise grinding, heating, or contacting in solution Compound 1 with a coformer under crystallization conditions, so as to form a cocrystal of Compound 1 with the coformer.

In some embodiments, a present cocrystal may be obtained by melting a Compound 1 and a coformer together and allowing recrystallization to occur.

In some embodiments, a present cocrystal may be obtained by mixing or grinding Compound 1 and a coformer together in the solid state, with or without solvent present.

In some embodiments, the cocrystal may be prepared by solution crystallization. In this method, Compound 1 and the coformer are separately dissolved in a solvent and the solutions combined. The cocrystal may then precipitate or crystallize as the solvent mixture is evaporated slowly. A cocrystal may also be obtained by dissolving the two components in the same solvent or in a mixture of solvents. Suitable solvents include, but are not limited to, polar protic or aprotic organic solvent including C₁-C₆ alcohols, C₃-C₁₂ alkanoic acid esters, C₃-C₇ alkyl ketones, cyclic and acyclic aliphatic ethers, nitroalkanes, alkanenitriles, lower alkaneamides, and halogenated hydrocarbons. Non-limiting examples of suitable solvents include methanol, ethanol, isopropanol, nitromethane, acetone, acetonitrile, ethyl acetate, dichloromethane, dimethylformamide, methyl tert-butyl ether, and mixtures thereof. In some embodiments, a present cocrystal may be obtained by stirring Compound 1 and a coformer together in the presence of a solvent. In some embodiments, the solvent is acetonitrile.

In some embodiments, the mixture of Compound 1, conformer, and solvent is heated. For example, the temperature may be above room temperature, such as about 25° C., about 50° C., about 75° C., about 100° C., or more, depending on compound solubility and solvent boiling point. In some embodiments, the temperature is at or near the boiling point of the solvent. In some embodiments, the temperature is about 50° C.

### Cocrystal Characterization

Cocrystals of the present disclosure may be detected by any suitable technique known in the art. Generally, the observation of physical properties of a solid (particularly its melting point) which differ from the physical properties of the starting materials (i.e., Compound 1 and the one or more coformers), is indicative of cocrystal formation. In some embodiments, the physical property is melting point or an X-ray diffraction pattern, such as a powder x-ray diffraction (PXRD) pattern or single crystal x-ray diffraction pattern. Crystalline forms may be reliably characterized by peak positions in the X-ray diffractogram, which produces a fingerprint of the particular crystalline form. The cocrystal diffraction pattern may be compared against a known crystal structure (e.g., Compound 1) to illustrate the presence of a different crystal form.

Melting point evaluation may be conducted by, for example, differential scanning calorimetry (DSC) or thermogravimetric analysis (TGA). Further characterization may be performed by conventional analytical methods, including, but not limited to, intrinsic dissolution profiles, equilibrium solubility, solid state NMR, Dynamic Vapor Sorption analysis (DVS), Fourier Transform Infrared (FTIR) spectroscopy, and Raman spectroscopy.

All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments. Any ranges cited herein are inclusive.

Aspects of the present invention are more fully illustrated with reference to the following examples. Before describing several exemplary embodiments of the invention, it is to be understood that the invention is not limited to the details of construction or process steps set forth in the following description. The invention is capable of other embodiments and of being practiced or being carried out in various ways. The following examples are set forth to illustrate certain aspects of the present invention and are not to be construed as limiting thereof.

### EXEMPLIFICATION

### Example 1. Upadacitinib 4-acetamidobenzoic acid cocrystal-acetonitrile solvate

Upadacitinib ((3 S,4R)-3-ethyl-4-(3H-imidazo[1,2-a]pyrrolo[2,3-e]pyrazin-8-yl)-N-(2,2,2-trifluoroethyl)pyrrolidine-1-carboxamide; 200 mg; 0.52 mmol) and 4-acetamidobenzoic acid (92 mg; 0.52 mmol) were charged to a vial. To the mixture of solids was added acetonitrile (800 μL). The suspension was mixed for approximately 24 hours at 25° C. The product was isolated by filtration.

An X-ray powder diffraction (“XRPD”) pattern of the product cocrystal was collected on a G3000 diffractometer (Inel corp., Artenay, France) equipped with a curved position sensitive detector and parallel beam optics. The diffractometer was operated with a copper anode tube (1.5 kW fine focus) at 40 kV and 30 mA. An incident beam germanium monochromator provides monochromatic Kα1 radiation (λ=1.540562 Å). The sample was prepared by spreading the sample powder in a thin layer on an aluminum sample holder and gently leveling with a glass microscope slide. The instrument was computer controlled using the Symphonix software (Inel Corp., Artenay, France) and the data analyzed using the Jade software (version 6.5, Materials Data, Inc., Livermore, CA). The aluminum sample holder was mounted on the rotating sample holder of the G3000 diffractometer and the diffraction data collected at ambient conditions.

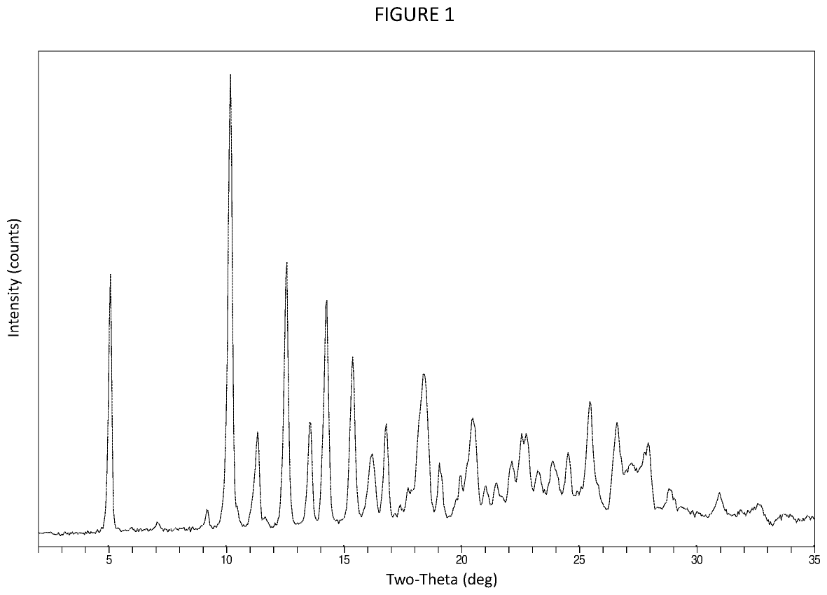

The X-ray powder diffraction pattern and XRPD peaks with relative intensity of the crystalline form are provided in FIG. 1 and Table 2, respectively.

TABLE 2
XRPD peak listing of upadacitinib 4-acetamidobenzoic
acid cocrystal acetonitrile solvate

| 2-Theta | Relative Peak |
|---|---|
| (degrees ±0.2) | Intensity (%) |
| 5.1 | 56.9 |
| 10.2 | 100.0 |
| 11.3 | 20.8 |
| 12.5 | 58.0 |
| 13.5 | 21.0 |
| 14.3 | 48.7 |
| 15.4 | 35.0 |
| 16.2 | 12.8 |
| 16.8 | 19.5 |
| 18.4 | 30.0 |
| 20.5 | 21.5 |
| 25.4 | 25.2 |

### Example 2. Crystallization of an upadacitinib 4-acetamidobenzoic acid cocrystal-acetonitrile solvate

The upadacitinib/4-acetamidobenzoic acid cocrystal of Example 2 (3 mg) and acetonitrile (300 μL) were charged to a vial and heated to 50° C. to dissolve the solids. The resulting solution was cooled to room temperature at a rate of approximately 5° C./hour. Crystals formed from the solution and were isolated. Single crystal X-ray diffraction data were measured using Mo Kα radiation λ=0.7107 Å The crystallographic profile of the crystalline form is summarized in Table 3.

TABLE 3
Crystallographic profile of upadacitinib 4-acetamidobenzoic
acid cocrystal acetonitrile solvate

| | |
|---|---|
| Wavelength (Å) | 0.7107 |
| Space group | P2₁2₁2₁ |
| Temperature (K) | 100 |
| a (Å) | 9.82 |
| b (Å) | 17.99 |
| c (Å) | 34.52 |
| α (°) | 90 |
| β (°) | 90 |
| γ (°) | 90 |
| V (Å³) | 6098.86 |
| Z | 8 |
| R-factor (%) | 6.95 |

### Example 3. Crystallization of an upadacitinib 4-acetamidobenzoic acid cocrystal-ethyl acetate solvate

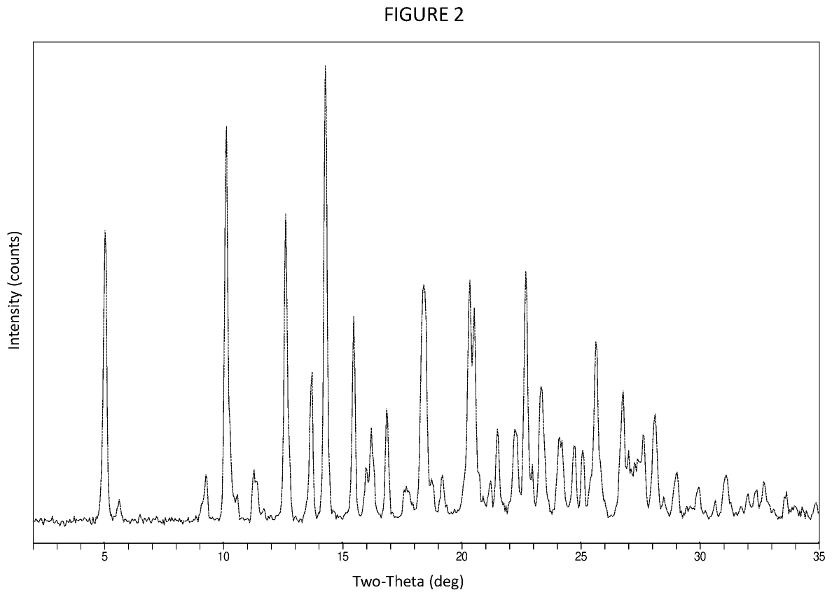

Upadacinitinib, 4-acetamidobenzoic acid cocrystal and ethyl acetate (15 volumes) were charged to a vial. The suspension was stirred at variable temperature from 5° C. to 50° C. followed by equilibration at 25° C. for approximately 24 hours. The product was isolated by filtration. A XRPD pattern was collected as in Example 2. The XRPD pattern and XRPD peaks with relative intensity of the crystalline form are shown in FIG. 2 and Table 4, respectively.

TABLE 4
XRPD peak listing of upadacitinib 4-acetamidobenzoic
acid cocrystal ethyl acetate solvate

| 2-Theta | Relative Peak |
|---|---|
| (degree) (±0.2) | Intensity (%) |
| 5.0 | 64.5 |
| 10.1 | 86.0 |
| 12.6 | 67.2 |
| 13.7 | 32.0 |
| 14.3 | 100.0 |
| 15.5 | 43.2 |
| 16.2 | 18.2 |
| 16.9 | 22.9 |
| 18.4 | 50.8 |
| 20.3 | 51.6 |
| 21.5 | 17.1 |
| 22.7 | 51.9 |

### Example 4. Crystallization of an upadacitinib 4-acetamidobenzoic acid cocrystal hydrate

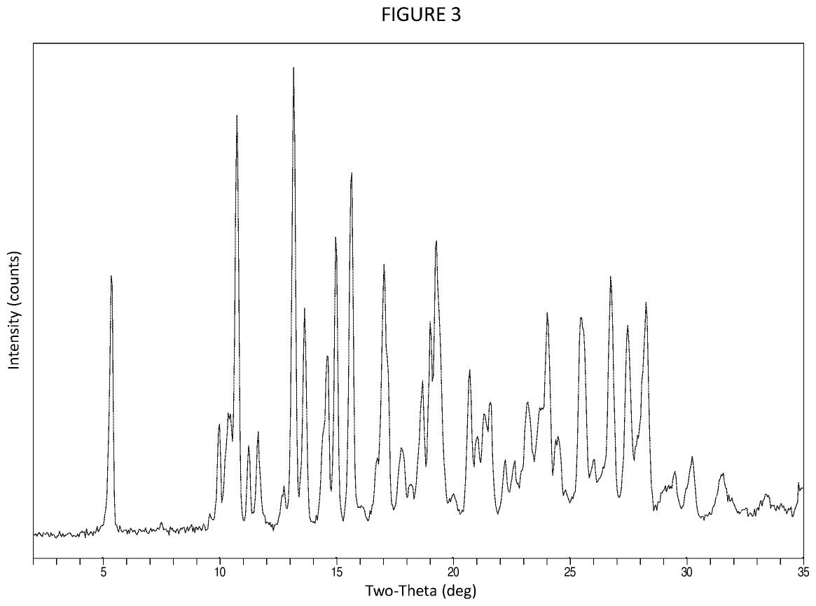

Upadacitinib (200 mg; 0.52 mmol) and 4-acetamidobenzoic acid (92 mg; 0.52 mmol) were charged to a vial. To the mixture was added acetonitrile (800 μL). The suspension was mixed for approximately 24 hours at 25° C. The product was isolated by filtration and dried at ambient conditions or under vacuum at 4° C. with a dry nitrogen purge. A XRPD pattern was collected as in Example 2. The XRPD pattern and peaks with relative intensity for the crystalline form are provided in FIG. 3 and Table 5, respectively.

TABLE 5
XRPD peak listing of upadacitinib 4-
acetamidobenzoic acid cocrystal hydrate

| 2-Theta | Relative Peak |
|---|---|
| (degrees ±0.2) | Intensity (%) |
| 5.3 | 56.3 |
| 10.0 | 23.0 |
| 10.7 | 85.8 |
| 11.2 | 17.5 |
| 11.6 | 20.6 |
| 13.2 | 100 |
| 13.6 | 45.8 |
| 14.6 | 34.8 |
| 15.0 | 60.2 |
| 15.6 | 74.3 |
| 17.0 | 53.2 |
| 18.7 | 27.6 |
| 19.3 | 59.3 |
| 20.7 | 31.0 |
| 24.0 | 40.8 |
| 25.5 | 37.3 |
| 26.7 | 45.2 |

### Example 5. Crystallization of an upadacitinib 4-acetamidobenzoic acid cocrystal hydrate

Upadacinitinib as the 4-acetamidobenzoic acid cocrystal was charged to a vial along with ethyl acetate (15 volumes). The suspension was stirred at variable temperature from 5° C. to 50° C. followed by equilibration at 25° C. for approximately 24 hours. The product was isolated by filtration and dried at ambient conditions or in vacuum oven at 40° C. with dry nitrogen purge. A XRPD pattern was collected as in Example 2. The XRPD pattern and XRPD peaks were identical to those for the upadacitinib 4-acetamidobenzoic acid cocrystal hydrate of Example 4.

### Example 6. Crystallization of an upadacitinib 4-hydroxybenzoic acid cocrystal-acetonitrile solvate hydrate

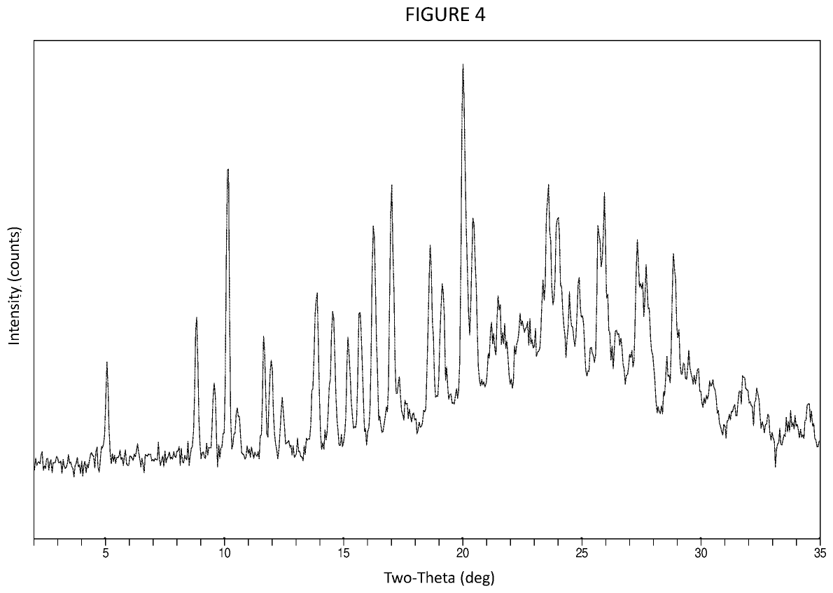

Upadacitinib (1000 mg) and 4-hydroxybenzoic acid (533 mg; a stoichiometric ratio of 1:1.5 upadacitinib:4-hydroxybenzoic acid) were charged to a vial. To the mixture acetonitrile (3 mL) was added. The suspension was mixed for approximately 24 hours at 25° C. The product was isolated by filtration. A XRPD pattern was collected as in Example 2. The XRPD pattern and XRPD peaks with relative intensity of the crystalline form are shown in FIG. 4 and Table 6, respectively.

TABLE 6
XRPD peak listing of upadacitinib 4-hydroxybenzoic
acid cocrystal mixed acetonitrile solvate/hydrate

| 2-Theta | Relative Peak |
|---|---|
| (degree ±0.2) | Intensity (%) |
| 5.1 | 31.2 |
| 8.8 | 41.5 |
| 9.6 | 26.3 |
| 10.1 | 75.7 |
| 11.6 | 37.1 |
| 12.4 | 23.0 |
| 13.9 | 47.1 |
| 14.5 | 42.8 |
| 15.2 | 36.9 |
| 15.7 | 42.6 |
| 16.2 | 62.6 |
| 17.0 | 72.0 |
| 18.6 | 58.2 |
| 19.1 | 49.2 |
| 20.0 | 100.0 |
| 20.4 | 64.4 |

### Example 7. Crystallization of an upadacitinib 4-hydroxybenzoic acid cocrystal-acetonitrile solvate hydrate

The upadacitinib 4-hydroxybenzoic acid cocrystal of Example 7 (15 mg) and acetonitrile (100 μL) were charged to a vial. Heat-cool cycles between 55° C. and 60° C. were applied. The resulting solution was slowly cooled to 40° C. and equilibrated at 40° C. for approximately 18 hours. Crystals were allowed to spontaneously cool to room temperature before isolation from the solution. Single X-ray diffraction data were measured using Mo Kα radiation λ=0.7107 Å. The crystallographic profile of the crystalline form is summarized in Table 7.

TABLE 7
Crystallographic profile of Upadacitinib 4-hydroxybenzoic
acid cocrystal mixed acetonitrile solvate/hydrate

| | |
|---|---|
| Wavelength (Å) | 0.7107 |
| Space group | P2₁2₁2₁ |
| Temperature (K) | 100 |
| a (Å) | 12.15 |
| b (Å) | 14.87 |
| c (Å) | 34.73 |
| α (°) | 90 |
| β (°) | 90 |
| γ (°) | 90 |
| V (Å³) | 6274.67 |
| Z | 8 |
| R-factor (%) | 6.57 |

### Example 8. Crystallization of an upadacitinib 4-hydroxybenzoic acid cocrystal-acetonitrile solvate hydrate

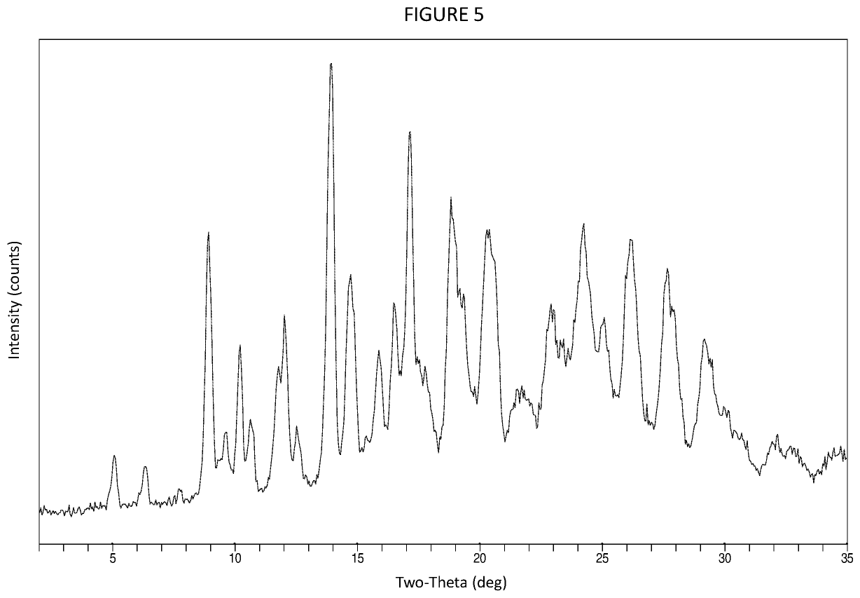

Upadacitinib (1000 mg) and 4-hydroxybenzoic acid (533 mg; a stoichiometric ratio of 1:1.5 upadacitinib:4-hydroxybenzoic acid) were charged to a vial. To the mixture acetonitrile (3 mL) was added. The suspension was mixed for approximately 24 hours at 25° C. The product was isolated by filtration and dried at ambient conditions or in a vacuum oven at 40° C. with dry nitrogen purge. A XRPD pattern was collected as in Example 2. The XRPD pattern and XRPD peaks with relative intensity of the crystalline form are shown in FIG. 5 and Table 8, respectively.

TABLE 8
XRPD peak listing of upadacitinib 4-
hydroxybenzoic acid cocrystal hydrate

| 2-Theta | Relative Peak |
|---|---|
| (degree ±0.2) | Intensity (%) |
| 5.1 | 17.6 |
| 6.3 | 15.3 |
| 8.9 | 64.6 |
| 10.2 | 40.8 |
| 10.6 | 25.2 |
| 12.0 | 47.2 |
| 13.9 | 100.0 |
| 14.7 | 55.6 |
| 15.9 | 39.8 |
| 16.4 | 49.7 |
| 17.2 | 85.7 |
| 18.8 | 71.9 |
| 20.3 | 65.1 |

### Example 9. Crystallization of an upadacitinib 4-hydroxy-3-nitrobenzoic acid cocrystal-acetonitrile solvate

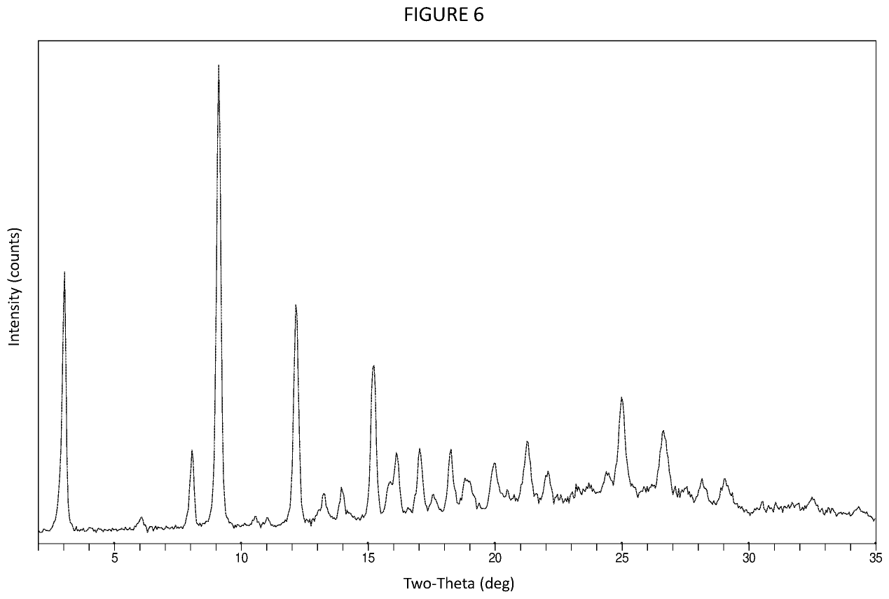

Upadacitinib (1000 mg) and 4-hydroxy-3-nitrobenzoic acid (942 mg; a stoichiometric ratio of 1:2 upadacitinib:4-hydroxy-3-nitrobenzoic acid) were charged to a vial. To the mixture acetonitrile (5 mL) was added. The suspension was mixed for approximately 24 hours at 25° C. The product was isolated by filtration. A XRPD pattern was collected as in Example 2. The XRPD pattern and XRPD peaks with relative intensity of the crystalline form are shown in FIG. 6 and Table 9, respectively.

TABLE 9
XRPD peak listing of upadacitinib 4-hydroxy-3-nitrobenzoic
acid cocrystal acetonitrile solvate

| 2-Theta | Relative Peak |
|---|---|
| (degree ±0.2) | Intensity (%) |
| 3.0 | 56.3 |
| 8.1 | 16.4 |
| 9.1 | 100.0 |
| 12.2 | 47.4 |
| 14.0 | 6.7 |
| 15.2 | 33.2 |
| 16.1 | 14.2 |
| 17.0 | 15.1 |
| 18.3 | 15.0 |
| 20.0 | 12.0 |
| 21.3 | 16.7 |
| 25.0 | 26.2 |

### Example 10. Crystallization of an upadacitinib 4-hydroxy-3-nitrobenzoic acid cocrystal-desolvated form

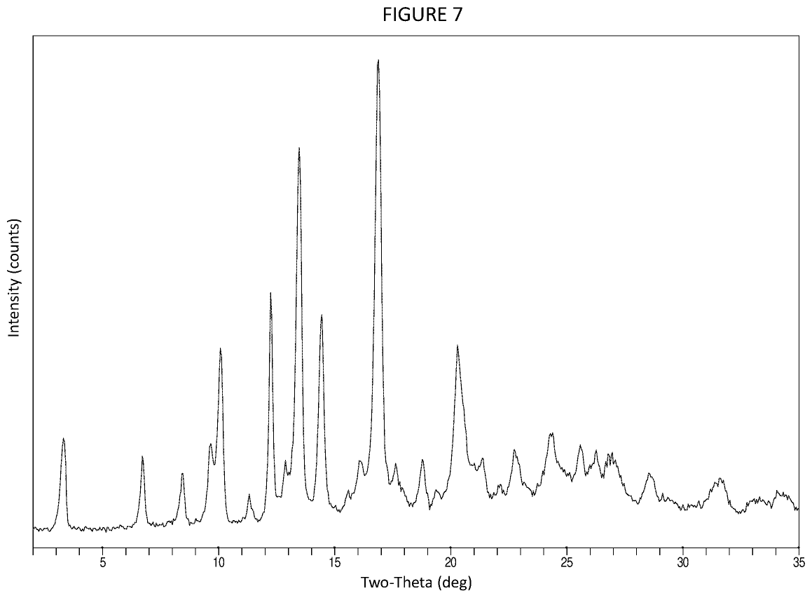

Upadacitinib (1000 mg) and 4-hydroxy-3-nitrobenzoic acid (942 mg; a stoichiometric ratio of 1:2 upadacitinib:4-hydroxy-3-nitrobenzoic acid) were charged to a vial. To the mixture acetonitrile (5 mL) was added. The suspension was mixed for approximately 24 hours at 25° C. The product was isolated by filtration and dried at ambient conditions or in vacuum oven at 40° C. with dry nitrogen purge. A XRPD pattern was collected as in Example 2. The XRPD pattern and XRPD peaks with relative intensity of the crystalline form are shown in FIG. 7 and Table 10a, respectively. XRPD data were measured using monochromatic Kα1 radiation λ=1.540562 Å. The crystallographic profile of the crystalline form is summarized in Table 10b.

TABLE 10a
XRPD peak listing of desolvated upadacitinib
4-hydroxy-3-nitrobenzoic acid cocrystal

| 2-Theta | Relative Peak |
|---|---|
| (degree ±0.2) | Intensity (%) |
| 3.3 | 21.9 |
| 6.7 | 15.6 |
| 8.4 | 10.9 |
| 10.1 | 38.3 |
| 12.2 | 50.4 |
| 13.4 | 76.8 |
| 14.4 | 41.3 |
| 16.9 | 100.0 |
| 18.8 | 11.2 |
| 20.3 | 33.3 |

TABLE 10b
Crystallographic profile of desolvated upadacitinib
4-hydroxy-3-nitrobenzoic acid cocrystal

| Space group | P2₁2₁2₁ |
|---|---|
| a (Å) | 17.3(2) |
| b (Å) | 21.4(5) |
| c (Å) | 52.1(5) |
| α (°) | 90 |
| β (°) | 90 |
| γ (°) | 90 |
| V (Å³) | 19288.5 |

### Example 11. Crystallization of an upadacitinib 3,4-dihydroxybenzoic acid cocrystal-acetonitrile solvate

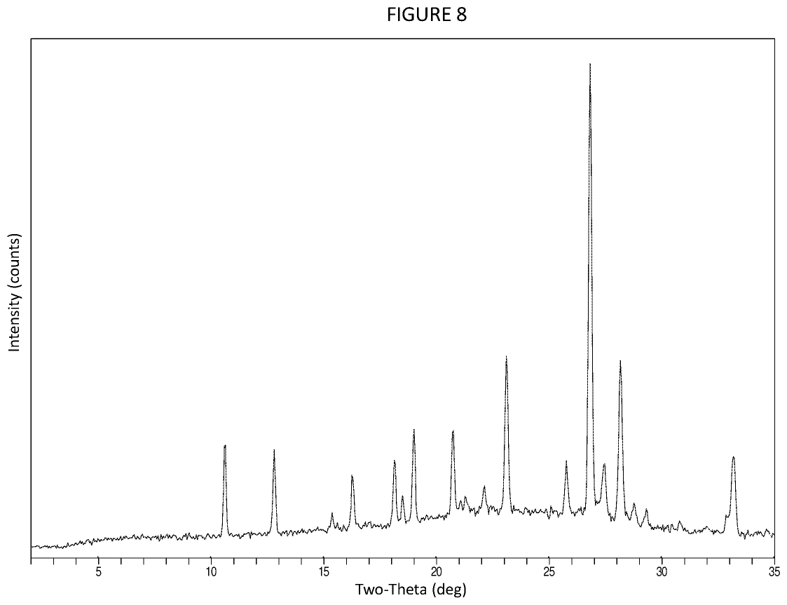

Upadacitinib (200 mg) and 3,4-dihydroxybenzoic acid (200 mg) were charged to a vial. To the mixture acetonitrile (0.4 mL) was added. The suspension was mixed for approximately 24 hours at 25° C. The product was isolated by filtration. A XRPD pattern was collected as in Example 2. The XRPD pattern and XRPD peaks with relative intensity of the crystalline form are shown in FIG. 8 and Table 11, respectively.

TABLE 11
XRPD peak listing of upadacitinib 3,4-dihydroxybenzoic
acid cocrystal acetonitrile solvate

| 2-Theta | Relative Peak |
|---|---|
| (degree ±0.2) | Intensity (%) |
| 10.6 | 23.5 |
| 12.8 | 18.5 |
| 16.3 | 11.6 |
| 18.1 | 13.3 |
| 19.0 | 19.8 |
| 20.7 | 18.7 |
| 23.1 | 34.4 |
| 25.8 | 11.5 |
| 26.8 | 100 |

### Example 12. Crystallization of an upadacitinib 3,4-dihydroxybenzoic acid cocrystal-hydrate

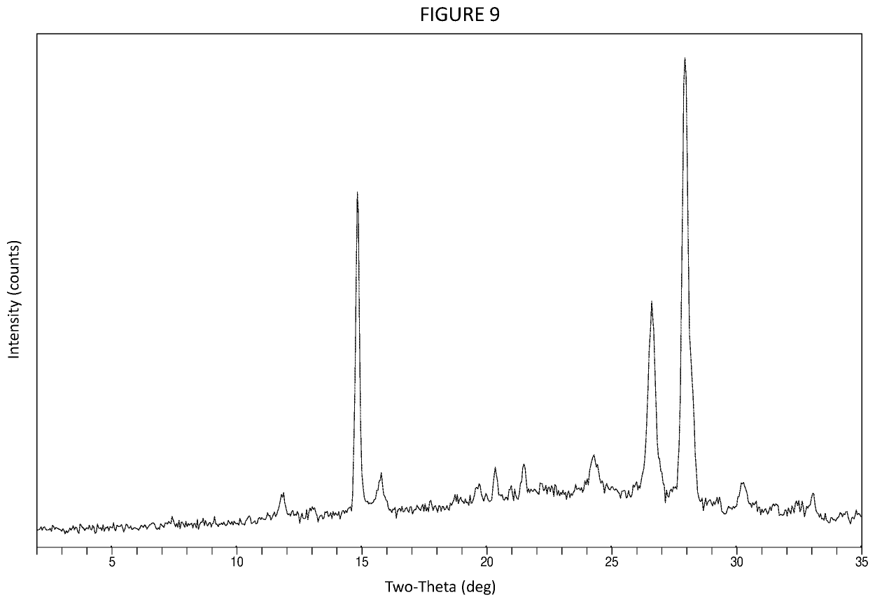

Upadacitinib (200 mg) and 3,4-dihydroxybenzoic acid (200 mg) were charged to a vial. To the mixture acetonitrile (0.4 mL) was added. The suspension was mixed for approximately 24 hours at 25° C. The product was isolated by filtration and dried at ambient conditions or under humidified conditions in a vacuum oven at 40° C. XRPD data were measured using monochromatic Kα1 radiation λ=1.540562 Å. The XRPD pattern and XRPD peaks with relative intensity of the crystalline form are shown in FIG. 9 and Table 12, respectively.

TABLE 12
XRPD peak listing of upadacitinib 3,4-
dihydroxybenzoic acid cocrystal hydrate

| 2-Theta | Relative Peak |
|---|---|
| (degree ±0.2) | Intensity (%) |
| 11.8 | 10.7 |
| 14.8 | 72.5 |
| 15.8 | 14.9 |
| 20.3 | 16.0 |
| 21.5 | 16.7 |
| 26.6 | 50.1 |
| 27.9 | 100 |

### Example 13. Crystallization of an upadacitinib 3,4,5-trihydroxybenzoic acid cocrystal

Upadacitinib (1000 mg) and 3,4,5-trihydroxybenzoic acid (437 mg; 1:1 molar ratio with upadacitinib) were charged to a vial. To the mixture acetonitrile (3 mL) was added. The suspension was mixed for approximately 24 hours at 25° C. The product was isolated by filtration.

### Example 14. Aqueous Solubility Determination of Upadacitinib Cocrystals

A solubility study was performed to determine the aqueous solubility for five of the upadacitinib cocrystals disclosed herein. Sufficiently sized samples of the solid cocrystals were added to individual vials along with a measured volume of water. The water-cocrystal mixtures were equilibrated at 37° C. by end-over-end tumbling for up to 24 hours. The amount of dissolved solid was determined for each sample. The solubility for each of the cocrystals is provided in Table 13.

TABLE 13
Aqueous solubility of upadacitinib and
upadacitinib cocrystals at 37° C.

| Upadacitinib Cocrystal | Solubility (mg/mL) |
|---|---|
| Upadacitinib Form I | 0.37 ± 0.004 |
| 4-hydroxy-3-nitrobenzoic acid cocrystal | 1.47 ± 0.01 |
| 4-acetamidobenzoic acid cocrystal | 2.53 ± 0.03 |
| 4-hydroxybenzoic acid cocrystal | 4.29 ± 0.05 |
| 3,4-dihydroxybenzoic acid cocrystal | 11.3 ± 0.2 |
| gallic acid cocrystal | 5.13 ± 0.05 |

### Example 15. Storage Stability Determination of Upadacitinib Cocrystals

A storage stability study was performed to determine the short term stability for each of the five upadacitinib cocrystals disclosed herein. Samples of each upadacitinib cocrystal were stored in an open dish at 40° C./75% relative humidity (RH) for up to one month. The chemical purity is provided in Table 14, which demonstrates that no meaningful change was observed for the stability attributes tested (purity and crystal form).

TABLE 14
Chemical purity of upadacitinib and upadacitinib cocrystals
after storage at 40° C./75% RH up to one month

| Upadacitinib Cocrystal | Purity (%) |
|---|---|
| Upadacitinib Form I | 100.2 |
| 4-hydroxy-3-nitrobenzoic acid cocrystal | 99.9 |
| 4-acetamidobenzoic acid cocrystal | 100.0 |
| 4-hydroxybenzoic acid cocrystal | 102.7 |
| 3,4-dihydroxybenzoic acid cocrystal | 103.9 |
| gallic acid cocrystal | 103.8 |

What is claimed is:

1. A cocrystal comprising (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (Compound 1) and a coformer, wherein the coformer is an aryl carboxylic acid.

2. The cocrystal of claim 1, wherein the aryl carboxylic acid is a substituted benzoic acid.

3. The cocrystal of claim 2, wherein the substituted benzoic acid has a structure of Formula (I) 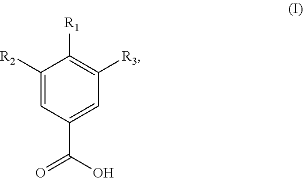
wherein:
R₁ is —NHC(O)CH₃ or —OH;
R₂ is —H, —OH, or NO₂; and
R₃ is —H, —OH, or NO₂.

4. The cocrystal of claim 3, wherein R₁ is —NHC(O)CH₃, and R₂ and R₃ are each H.

5. The cocrystal of claim 3, wherein R₁ is OH, and R₂ and R₃ are each H.

6. The cocrystal of claim 3, wherein R₁ is OH, R₂ is —OH, and R₃ is H.

7. The cocrystal of claim 3, wherein R₁, R₂ and R₃ are —OH.

8. The cocrystal of claim 3, wherein R₁ is OH, R₂ is —NO₂, and R₃ is H.

9. The cocrystal of claim 1, wherein a molar ratio of Compound 1 to coformer is from about 5:1 to about 1:5.

10. The cocrystal of claim 9, wherein the molar ratio is from about 2:1 to about 1:2, or from about 1:1.5 to about 1.5:1.

11. The cocrystal of claim 9, wherein the molar ratio is about 1:1.

12. The cocrystal of claim 1, which is a solvate.

13. The cocrystal of claim 12, wherein the solvate comprises acetonitrile.

14. The cocrystal of claim 13, further comprising water.

15. The cocrystal of claim 1, having one or more of reduced aqueous solubility, reduced dissolution, enhanced bioavailability, enhanced stability, increased Cₘₐₓ, increased or decreased Tₘₐₓ, increased half-life, increased AUC, enhanced processability, and reduced hygroscopicity, relative to Compound 1 as a free base or a salt, including solvates, hydrates, and polymorphs of any thereof.

16. A cocrystal comprising (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide (Compound 1) and 4-acetamidobenzoic acid in a molar ratio of approximately 1:1.

17. The cocrystal of claim 16, which is an acetonitrile solvate.

18. The cocrystal of claim 17, which is a hydrate.

19. The cocrystal of claim 17, having an x-ray powder diffraction pattern characterized by peaks at 5.1±0.2, 10.2±0.2, and 12.5±0.2 degrees two theta when measured at about 25° C. with monochromatic Kα1 radiation λ=1.540562 Å.
